(19) United States
(12) Patent Application Publication
(10) Pub. No.: US 2009/0258386 A1
(43) Pub. Date: Oct. 15, 2009

(54) METHODS FOR SOLID MUTAGENESIS AND SEMI-SOLID FLUID MUTAGENESIS FERMENTATION AND PURIFICATION OF LIPID SOLUBLE VITAMINS AND NUTRIENTS

(75) Inventor: DANIEL R. DEBROUSE, Oklahoma City, OK (US)

Correspondence Address: NYEMASTER, GOODE, WEST, HANSELL & O'BRIEN, P.C., 700 Walnut Street, Suite 1600, Des Moines, IA, 50309-3899, US

(73) Assignee: BIOSYM TECHNOLOGIES OF IOWA, L.L.C., WEST DES MOINES, IA (US)

(21) Appl. No.: 12/100,134

(22) Filed: Apr. 9, 2008

(51) Int. Cl. C12P 23/00; C12P 1/00; C12P 7/66; C12P 1/04

(52) U.S. Cl. 435/67; 435/41; 435/170; 435/133

(57) ABSTRACT

According to the invention, Applicants have demonstrated methods for improving industrial biosynthesis of lipid soluble vitamins and nutrients. Applicants have also provided methods for cost-efficient and commercially-viable chemotherapeutic biosynthesis and purification. This invention provides novel methods for both solid mutagenesis and semi-solid fluid mutagenesis fermentation and the purification of lipid soluble vitamins and nutrients and increasing fermentation solid yields.

### FIELD OF THE INVENTION

This invention relates to methods for both solid mutagenesis and semi-solid fluid mutagenesis fermentation and the purification of lipid soluble vitamins and nutrients. In particular, this invention relates to methods for improving industrial biosynthesis of lipid soluble vitamins and nutrients, as well as providing methods for cost-efficient and commercially-viable chemotherapeutic biosynthesis and purification.

### BACKGROUND OF THE INVENTION

The present invention relates to solid mutagenesis (SM) and semi-solid fluid mutagenesis (SSFM) fermentation and purification of lipid soluble vitamins and nutrients. The present invention solves numerous problems found in the technological and industrial processes for biosynthesis of lipid soluble vitamins and nutrients. The field of environmental microbiology and its applicable commercial industries has failed to develop safe, effective and cost-efficient methods for fermentation and purification of lipid soluble vitamins and nutrients.

An exemplar bionutrient of importance is lycopene, a carotenoid phytochemical pigment found widely distributed throughout nature, which is a highly desirable molecule to harvest for commercial use. Lycopene is expressed in botanical sources, microscopic photosynthetic bacteria, and fungi. It is known as psi,psi-carotene and is a terpene molecule assembled from 8 isoprenoid units. The molecule is highly unsaturated containing 13 conjugated carbon-carbon double bonds which are ultimately responsible for the molecules pigmentation. For example, each double bond reduces the energy required for electrons to transition to higher energy states allowing the molecule to absorb visible light of progressively larger wavelength and thus the molecule absorbs most of the visible spectrum and appears red.

The health-boosting antioxidant properties of lycopene make the molecule extremely useful for both the pharmaceutical and neutraceutical industries. It is the most potent and prevalent of the carotenoid antioxidants found in the human body and may play major roles in both prostate and cardiac health. However, traditional developmental and implementation schemes for the provision of lycopene for consumer supplementation have failed to provide adequate supplies of the molecule.

The existing industrial practices employed for nutrient biosynthesis and harvest require the use of hazardous solvents for nutrient extraction from the reservoir and purification. The primary extraction schemes to effect the purification of a nutrient such as lycopene from a natural reservoir, include extraction under reflux, soaponification and supercritical fluid extraction techniques. Extraction under reflux and soaponification require lycopene extraction from a biomass with the use of some combination of dichloromethane, ethyl acetate, toluene, benzene, acetonitrile, chloroform, and both methyl and ethyl alcohol. The use of such extraction techniques involves harsh and potentially carcinogenic solvents. Any such refining and purification processes employed result in detectable traces of these solvents upon chromatography analysis of the end product dispersions supplied to retail markets, making the products undesirable due to the numerous health risks involved in polluting the consumer population with solvent residues. Alternatively, the methods of the present invention encompass all natural mutation processes, resulting in the elimination of the need for harsh and potentially carcinogenic solvents, providing safer working conditions and other benefits associated with an all-natural mutation process.

As a result, some have attempted to utilize subcritical and supercritical fluid extraction methods to harvest carotenoids. Both sub and supercritical extraction processes use an inert gas, normally carbon dioxide, to effect the fractionation of a biologically active molecule from a natural biomass without the use of hazardous solvents. However, these techniques have not achieved widespread industrial implementation due to cost and optimization failures. Notably, for supercritical fluid extraction, extreme pressures and critical conditions are applied which are not only dangerous to the technician, but require highly skilled personnel. Additionally, sub critical fluid extraction fails to remove undesirable molecules and lipid impurities from the extracted bionutrient.

Accordingly, it is an objective of the invention to provide a method for chemotherapeutic purification innovations to eradicate the practice of using hazardous solvents in the processing of bionutrients.

Therefore, it is an objective of the present invention to utilize biomimicry to develop superior mechanisms to bioactive production, purification, and dissolution.

It is another objective of the present invention to utilize biomimicry to enhance biosynthesis pathways and purification for industrial implementation.

It is yet another objective of the present invention to develop methods of SSFM fermentation to remove all impurities from an extracted bionutrient.

A further objective of the invention is a method of solid mutagenesis fermentation and the purification of lipid soluble vitamins and nutrients.

A further objective of the invention includes a method of semi-solid fluid mutagenesis fermentation and the purification of lipid soluble vitamins and nutrients.

Yet another objective of the invention includes methods for improving industrial biosynthesis of lipid soluble vitamins and nutrients.

A still further objective of the invention includes methods for cost-efficient and commercially-viable chemotherapeutic biosynthesis and purification.

### SUMMARY OF THE INVENTION

This invention provides novel methods for both solid mutagenesis and semi-solid fluid mutagenesis fermentation and the purification of lipid soluble vitamins and nutrients by analyzing a host source of a lipid-soluble vitamin or nutrient for genetic, biochemical, physical, and propagation characteristics; determining a preferred form of extended mutagenesis for the host; exposing the host source to fermentation; generating a hypersensitive strain of the host source; and increasing fermentation solid yields. According to the invention, Applicant has demonstrated methods for improving industrial biosynthesis of lipid soluble vitamins and nutrients. Applicant has also provided methods for cost-efficient and commercially-viable chemotherapeutic biosynthesis and purification.

### BRIEF DESCRIPTION OF THE DRAWINGS

FIG. 1 shows the bioreactor in which SM fermentation is carried out.

FIG. 2 shows the process for the production of macro-nutrient spheres.

FIG. 3 shows the enzymatic pathway and corresponding genes in plants in carotenoid biosynthesis.

FIG. 4 shows the two-step process of an electrophilic addition reaction occurring on a lycopene molecule.

FIG. 5 shows the mathematical expression of biosymbiotic fermentation.

### DETAILED DESCRIPTION OF THE PREFERRED EMBODIMENT

SM and SSFM fermentation represent technological leaps in the industrial biosynthesis of lipid soluble vitamins and nutrients. Nutrients are classified in this application as any substance used by an organism and taken in from an environmental source. For example, non-autotrophic organisms usually acquire nutrients by the ingestion of foods. Organic nutrients most often include carbohydrates, fats, proteins or amino acids, and vitamins. Inorganic chemical compounds such as minerals, water and oxygen may also be considered nutrients.

The two fermentation techniques of the present invention represent significant advances particularly within the field of environmental microbiology. Microbiologists have well established the interlinking-nutrient co-operations and competitions occurring between microbes within a local ecosystem. Such interactions have at their molecular bases a genetic response to the presence of a biological “marker” from a local resident or transient organism. Over persistent exposures such genetic triggers can lead to a stable mutation within competitor organisms, an example of microscopic evolution.

Genomic mutations are less frequent than hypersensitive states of proliferation and expression brought on by the persistent exposure to “promoter triggers.” Genomic mutations generally arise under conditions of extreme “marker” exposure, leading to cell death and the depletion of a particular strain of microorganism. There are two separate mechanisms for inducing a hypersensitive state of proliferation and expression in a host source: (1) a stable mutation or adaptation in the genome of a microorganism through an alteration in the DNA base sequence of a particle gene or set of genes as a result of prolonged exposure to an environmental stress or biochemical “marker” which endangers the survival of the species; and (2) promoter triggering brought about through an intercellular response to the presence of an extracellular biomolecule produced and secreted or displayed within the phospholipid membrane of a competing organism, or through the depletion of one or more vital nutrients from the local nutritional pool.

When a “promoter trigger” causes the disassociation or association of a repressor protein to the promoter region of a specific gene or operon, it causes the transcription or transcriptional inhibition of the corresponding messenger RNA product and thus its coded functional protein. Therefore, over extended exposures of a “promoter trigger” may bring about a stable mutation within the DNA sequence a repressor protein recognizes or within the DNA sequence encoding the repressor protein itself.

An alternative to alterations at the cellular level are improvements to the industrial biosynthesis of lipid soluble vitamins and nutrients based on extraction from a bionutrient source. Accordingly, a primary objective of the present invention is to utilize biomimicry to develop superior methods for bioactive production, purification, and dissolution, as well as enhance biosynthesis pathways and purification for industrial implementation. Biomimicry incorporates both a philosophy and practice of observing a micro-environment in nature. For example, observing the microbial interactions occurring in soil, and the molecular basis of the symbiotic and stress interactions occurring between the complex pool of present microorganisms, related technology can be applied to laboratory processes in a novel simulated selection to mimic, in an artificial way, such molecular activities. Through the emulation and modeling of natural methods and occurrences, improved and efficient methods and processes are developed to overcome existing problems in the art.

In the practice of SM and SSFM fermentation, a host source of a particular lipid soluble vitamin or nutrient of biomedical importance, botanical or microbial, must be identified. Those of ordinary skill in the art have failed to achieve methods for generating increased fermentation solid yields over existing industrial yields in order to produce more cost-effective means for generating commercial supplies of various fermentation solids. The present invention solves the existing problem in the art by providing methods for increasing the generation of fermentation yields through SM and SSFM fermentation and purification of lipid soluble vitamins and nutrients.

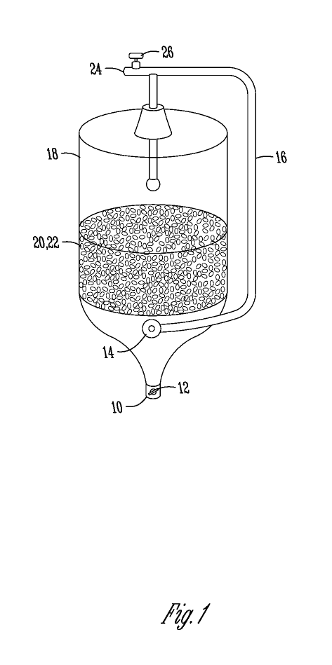

SSFM fermentation is a fermentation process carried out in a novel formulation wherein the nutrient constituents produce a medium that ranges from essentially 5 to 85 percent solids. Preferably, the medium ranges from essentially 30 to 80 percent, and even more preferably essentially 60 percent solids, providing an optimized environment for an organism's adaptation to a hypersensitive state of bioexpression in a non-genetically engineered manner. The SSFM fermentation is carried out in a novel bioreactor of FIG. 1.

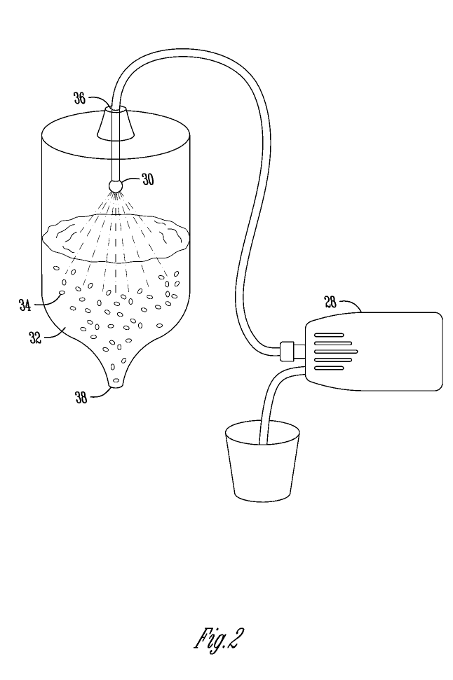

SM fermentation is a fermentation process carried out in a novel formulation wherein the nutrient constituents produce a medium that ranges from essentially 85 to 100 percent solids. Preferably, the medium ranges from essentially 90 to 100 percent, and even more preferably essentially 96 percent solids, providing an optimized environment for an organism's adaptation to a hypersensitive state of bioexpression in a non-genetically engineered manner. The fermentation is carried out in a novel bioreactor on novel macro-nutrient spheres as shown in FIG. 2.

SM and SSFM fermentation techniques are used for prolonged proliferative mutagenesis of microorganisms. In one embodiment, the environmental yeast Phaffia rhodozyma undergoes SM and SSFM fermentation to generate the biosynthesis of astaxanthin. Phaffia is most commonly utilized within the pharmaceutical and neutraceutical industries for the commercial production of astaxanthin. Traditional commercial methods of biosynthesis of astaxanthin involve the yeast mutation with either a reagent such as ethyl methane sulphonate, N-methyl-N-nitro-N-nitrosuguanidine or some other nucleotide base analogue, or through UV irradiation as described in U.S. Pat. No. 5,648,261 incorporated herein by reference in its entirety. Such reagents are selective and limited in its ability to mutate the yeast, targeting a localized region with the carotenoid operon resulting in an increase in the transcription of those genes involved in the biosynthesis of astaxanthin.

In another embodiment of the present invention, the methods of SM and SSFM fermentation are applied to the mutagenesis and subsequent fermentation of carotenoid expressing microorganisms. For the carotenoid astaxanthin, the wild-type yeast Phaffia rhodozyma underwent SSFM in a high viscosity medium of a solid agar, over the course of 8 months and adapted to a peptone substrate in the presence of a natural chemical mutagen to change cell size, rate of proliferation and astaxanthin expression. Then the organism was inoculated on peptone agar in the SM fermentation system, a high viscosity medium of a solid agar, and incubated for 48 hours, resulting in the harvest of fermentation solid level of 3.70 g per 25.0 g of medium (approximately 14.8% w/v of the fermentation medium) with an astaxanthin concentration of 70,000 ppm, preformed by HPLC analysis. The novel methods produced the unexpected results of a profound increase in fermentation solid yields over the current industrial reported yields in the fermentation of Phaffia for the biosynthesis of astaxanthin, where the yields at best are about 6% w/v of the fermentation medium and an astaxanthin concentration of only 4,000 ppm.

In yet another embodiment of the present invention, the yeast Rhodotorula glutinis, a predominant expresser of beta-carotene, underwent a similar two-step mutagenesis fermentation process and also resulted in a greater than 50% increase in fermentation solids and carotene concentration compared to the most efficiently reported industrial strains utilized by one of ordinary skill in the art. SM and SSFM fermentation methods were also utilized to yield vitamin K2 MK-7 from the bacterium Bacillus subtilis. The organism was adapted to a soy protein extract in the SM fermentation system in the presence of a natural chemical mutagen for six months and then inoculated into dextrose soy medium in the SSFM system. Yet again, the yields of fermentation solids far exceeded those obtained from the current industrial applications.

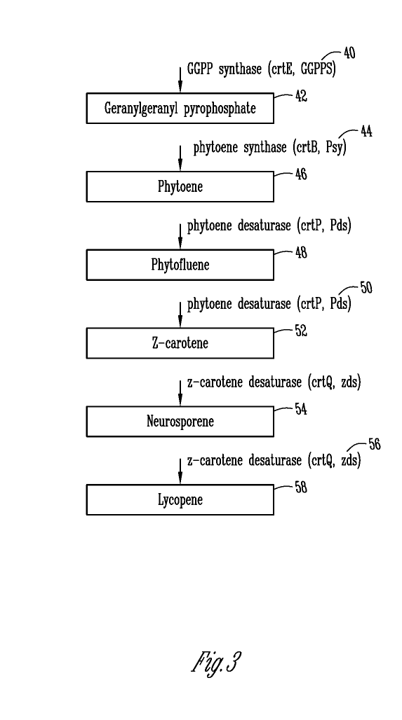

Similarly, yet another embodiment of the present invention involving the biosynthesis of lycopene from a Paracoccus bacterial species yielded a similar increase in fermentation solids and lycopene concentration. The biosynthesis of lycopene in nature is a highly regulated process. In higher plants there are a minimum of four genes involved in the biosynthesis of this molecule, in contrast to only three involved gene products in the photosynthetic bacteria and fungi (FIG. 3). In higher plants the biosynthetic pathway leading to lycopene expression is initiated by geranylgeranyl pyrophosphate synthase (GGPPS) 40 through its biosynthesis of geranylgeranyl pyrophosphate 42 which is in-turn converted to phytoene 46 by the second enzyme involved in the biosynthesis, phytoene synthase 44. Next phytoene is converted to phytofluene 48 and then to z-carotene 52 through the activity of the phytoene desaturase enzyme 50. Finally, z-carotene is converted to neurosporene 54 which is then quickly desaturated to lycopene 58 by the final enzyme expressed in this biosynthetic pathway, z-carotene desaturase 56.

Lycopene is an organic aliphatic molecule often called a conjugated diene, and is expected to undergo chemical reactions common to alkenes, such as electrophilic addition reactions (addition of acidic reagents). The most characteristic feature of the lycopene molecule, as with any alkene, is the carbon-carbon double bond. It is the functional group of the lycopene structure and determines the characteristic reactions the molecule undergoes. Understanding of the characteristic reactions allows manipulation of the various methods for biosynthesis and extraction of the molecule, in order to prevent the occurrence of the undesirable additions throughout the industrial manufacture and purification of lycopene. Preventing such additions requires creating slightly alkaline fermentation and extraction pools with low percent sodium hydroxide mixes to effectively inhibit electrophonic additions.

Chemical analysis of the lycopene structure indicates that the electrophilic addition reactions that occur at the carbon-carbon double bonds of the lycopene structure are rate dependent upon lycopene and acidic reagent concentration. The addition of HZ to the carbon-carbon double bond of the lycopene molecule involves the transfer of a proton to one of the many electron rich carbons. Lycopene is a base and accepts protons to a significant degree only from strong acids. Even the familiar acetic acid CH₃COOH, is not strong enough and alone does not read with the lycopene molecule.

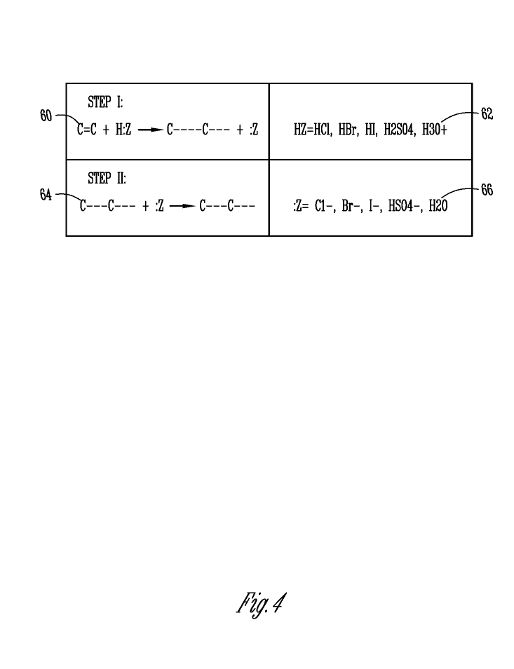

The carbon-carbon double bonds that make up the lycopene molecule consist of a strong O bond and a weak ̂ bond. The typical reactions involve breaking the weak bonds and forming two strong bonds in its place. Only acidic reagents seeking electron donors react with the electron rich carbon-carbon double bonds of the lycopene molecule, referred to as electrophilic addition reactions (FIG. 4). Step I involves the transfer of hydrogen ion from :Z to the available electrons of the double bond resulting in the formation of a carbocation (transfer of a proton from one base to another) 60, whereas Step II involves the base of the acid combining rapidly with the intermediate and highly reactive carbocation 64.

Step I is the slow difficult rate-dependent step of the process, its rate largely or entirely controls the overall rate of addition. This step involves attack by an acidic, electron-seeking reagent, that is, an electrophilic reagent, and hence the reaction is an example of electrophilic addition. The electrophile can be any kind of electron deficient molecule and could involve the addition of hydrogen chloride, sulfuric acid, or water to identify a few 62. However, when the carbocation combines with water it forms a protonated alcohol, which in a subsequent reaction releases a hydrogen ion to another base to form the alcohol 66, illustrating the equilibrium shift in favor of the alcohol by the high concentration of water.

In determining appropriate host sources of a particular lipid soluble vitamin or nutrient of biomedical importance, the availability and cost to obtain such sources must practically be considered. For example, lycopene biosynthesis requires identification of various natural reservoirs of high enough concentrations of the carotenoid. Necessarily, this involved sampling natural reservoirs and processing and analyzing the lycopene concentration, primarily accumulating within chromoplasts, by HPLC (Table 1).

TABLE 1

| | | % Lycopene |
|---|---|---|
| Biomass | % Lycopene Wet Weight | Dry Weight |
| Full Red Tomato Paste | 42.0 mg/100 g | 102.0 mg/100 g |
| 26% 170430 Tomato Paste | 57.0 mg/100 g | 117.9 mg/100 g |
| Tomato Skin | 63.6 mg/100 g | 323.24 mg/100 g |
| Watermelon | 10.3 mg/100 g | 15.4 mg/100 g |
| Papaya | 3.0 mg/whole fruit | NA |
| Grapefruit | 2.9 mg/whole fruit | NA |
| Paracoccus dentrificans | 80 mg/100 g | 296.3 mg/100 g |
| Paracoccus marcusii | 63 mg I100 g | 233.1 mg/100 g |
| Jackfruit | 276.37 mg/100 g | 484.86 mg/100 g |
| Bell pepper | 167.63 mg/100 g | 349.23 mg/100 g |

The jackfruit and bell pepper express lycopene concentrations in far excess of any other natural botanical reservoir analyzed and would be well suited for SM and SSFM fermentation. However, the feasibility of using other natural reservoirs, such as the photosynthetic bacteria presented itself as the most convenient source of lycopene. The bacterium Paracoccus presents as an ideal candidate for SM and SSFM fermentation as it is capable of being more readily “hypersensitized.”

“Super-strains” of various sapoids, the natural decomposers, are capable of simulating the six week decomposition process of various botanical sources of lycopene in a matter of hours or days alone or in combination. Therefore, SSFM methods can be used to “hypersensitize” a fungal or bacterial sapoid species for the natural decomposition of the Paracoccus species and the extraction of biosynthesized lycopene. A bacterial sapoid capable of excreting a lysogenic enzyme, upon its fermentation within the Paracoccus pool, readily disrupts the bacterial cells releasing the lycopene-filled chromoplasts. This in turn disrupts the freed lycopene pooled to the surface of the medium for easy removal and purification and generating a significant increase in the rate of decomposition and lycopene extraction. Additional soil sapoids include Fusarium, Trichoderma, Penicillum, Aspergillus, Bacillus subtilis, Bacillus licheniformis, Bacillus pumilius, Leuconostoc mesenteroides, Chrysosporium pannorum, Rhizobium, Bradyrhizobium, and other related species.

Upon determining the host source for a bionutrient for use with the methods of SM and/or SSFM fermentation, it must first be determined which of the techniques is best suited for extended mutagenesis of a particular nutrient source. The applicable genetic, biochemical, physical and propagation characteristics of the nutrient source are reviewed to determine the most appropriate system for the desired effect. For example, the desired outcome may be to adapt the yeast Phaffia rhodozymna to become an organism capable of generating a more cost-effective nutrient pool and a “hypersensitive” strain of the source to express the desired nutrient, astaxanthin, in significantly greater concentrations compared to strains derived through traditional mutagenesis.

The selected nutrient source is then first exposed to the SSFM or SM process. The Phaffia yeast, for example, was first exposed to a SSFM process (as identified in Example 1), including: preparing an SSFM agar source, inoculating the yeast, vortexing and incubating with agitation. The vortex agitates test tubes, without separating its contents. Streak plates are prepared from available cultures approximately every 30 days, recording measurements including, cell survival, pigmentation, and morphology. The SSFM process for Phaffia indicated, via microscopic examination, that until the eighth month, surviving cells remained albino and appeared as large oval cells. At the eighth month, however, one of the tubes produced a bright purple colony appearing within 8 hours from the time the streak was prepared. Upon microscopic examination the cells appeared minute in size, smaller than that of most bacteria, but still maintained their oval morphology. Once more upon drying, extraction, and HPLC analysis of the cell solids, a concentration of 70,000 ppm astaxanthin was found present.

The resultant increase in solid yields indicates that some transcriptional control within the carotenoid operon had been mutated, in addition to a genetic alteration in the proliferative cycle of the organism. The species genetic code was altered in such a manner as to redirect cellular activity to the concentration of astaxanthin pooled chromoplasts at the expense of cell mass. This produces a “hypersensitive” state of propagation and expression, achieving the proposed goals of the present invention, as illustrated by a comparison of the mutated organism with the currently known Phaffia mutants. There are no known express pigments within the growth phase of the cell cycle as demonstrated by the “hypersensitive” strain resultant from the methods of the present invention. To be certain, pulse field gel electrophoreses was used to compare this mutated organism to all known strains of Phaffia, at the genetic level, and the species was found to be more genetically compatible with the genus Rhodotorula.

Notably, the microscopic and biochemical analyses of the microorganisms adapted by SM and SSFM fermentation illustrate that the cells are many times smaller than those of the wild-type species. The genomes are mutated to immediately stimulate transcription of the genes involved in bioactive expression within the growth phase of the cell cycle. Furthermore, it appears as if the transcriptional controls operating in these operons of the wild-type organisms have been eliminated all together. In every instance, maximum cellular proliferation, bioactive expression and concentration occur within a 48 hour period. For example, in the case of astaxanthin from Phaffia, 8 hours after inoculation a thick almost tissue-like growth appears bright purple in coloration.

The development of SM fermentation resulted from the need to adapt the Phaffia strain to a fluid agar medium. Various formulation attempts resulted in a consistent, small amount of organism growth in a “tissue-like” manner within the mediums. The SM fermentation was subsequently adapted from the initial bench scale to the commercial process. Application of the SM fermentation method yields preferential results for the commercial propagation of Phaffia rhodozyma for the biosynthesis and recovery of astaxanthin, in comparison to SSFM.

SM fermentation is carried out in a conical bioreactor 18 which provides an attachment nutrient matrix for the propagation of SSFM “hypersensitized” microbes (FIG. 1). The first step in SM fermentation involves the production of nutrient macro-spheres 34 through a process of cold water atomization of an agar nutrient pool. The agar nutrient macro-sphere for a “hypersensitized” strain of Phaffia is composed of sterilized 3.0 g/L maltose extract, 5.0 g yeast extract, 3.0 g/L dextrose and 20.0 g/L agar in an atomization vessel. After the sterilization process, the hot solution is rapidly sprayed using an atomizer 28 and spray gun 36 through an atomization tip 30 into a pool of ionized cold water 32. Upon contacting the surface of the water, macro-spheres of agar nutrient are formed 34 (FIG. 2). The macro-spheres can be released through the outlet valve 38.

The macro-spheres 20 are loaded into the SM bioreactor 18 and the system is charged with a pool of SSFM “hypersensitized” microbes 22 prior to activating the fermentation process. Notably, the SM bioreactor system can be operated in an anaerobic or aerobic state, under either a static or dynamic state, under any level of required carbon dioxide and may even be run under a flow of nitrogen gas. The system is fully equipped with computerized monitoring devices for the precise measurement and control of temperature, pH, carbon dioxide concentration, oxygen concentration, lighting cycles, incubation period and nutrient concentration and replenishment. Specifically, the bioreactor 18 contains a water inlet 24 and shut-off valve 26, circulator 16, submersible pump 14, shut-off valve 12 and outlet 10. In addition, the bioreactor is completely automated, requiring only computerized technician interference. At the end of a SM fermentation period, the system is rapidly discharged by purging with either oxygen or nitrogen gas and thus the separation of the fermentation solids from the macro-nutrient spheres.

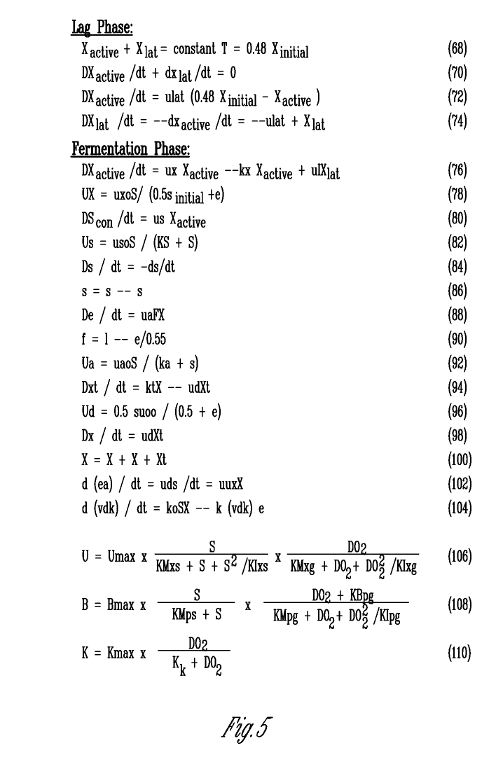

The methods for SM and SSFM fermentation of a bionutrient are capable of mathematical expression using the model of FIG. 5 to express the parameters of the SM and SSFM fermentation methods. The model is sufficient to make predictions based upon substrate variance and changes in temperature and in oxygen concentration. There are two consecutive periods during the fermentation: lag 68-74 and active fermentation 76-110 (for each genus of bacteria involved there must be a separate calculation). Similarly, each bacterium consists of three types of cells: lag, active, and dead. The number of lag cells being smaller in comparison to the other types. The three different cell types in each group genus may be expressed within the same equations capable of predicting and expressing optimal parameters for fermentation.

During the lag phase 68-74 very little ethanol is produced, lag cells are transformed to active cells, and the biomass is settled to the bottom. Similarly, little lycopene (or other applicable bionutrient extracted) produced during the lag phase.

During the fermentation phase 76-110, suspended active bacterial concentration increases and decreases and temperature relations in regard to the gradient of trajectory and carbonic activity. The evolution of sugars is described in equations 80-86. Alcohol concentration in dependent on temperature and ethanol production rates and limits, and active bacterial concentrations are described with equations 76 and 78 and ethanol rates with 88. The fermentation in mathematical sense should focus on 35 points.

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, processes and examples described in the description of the invention are illustrative only and not intended to be limiting to the scope of the invention in any manner. Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims.

### EXAMPLES

This invention can be better understood by reference to the following examples. The present invention is not limited in terms of scope based on the following examples which are provided only for purposes of illustration of embodiments of the invention. It will be apparent to those skilled in the art that other embodiments of the invention may be practiced without departing from the spirit and scope of the invention, which is defined by the appended claims.

### Example 1

The preparation of 100 mL of SSFM agar includes the following: 1.5 g peptone water (Becton Dickinson), 0.5 g agar (Becton Dickinson), 0.016 g of Citro Bio (Sarasota Fla.) as mutagen, and 100 mL of distilled water. The mixture is boiled for 2 minutes prior to its distribution into 10 separate 10 ml/mL test tubes, sterilized with steam for 30 minutes, cooled to room temperature and then inoculated with 1,000,000 CFU of wild-type Phaffia rhodozyma. Tubes are then vortexed for 60 seconds and incubated under 1700 lumens of UV light at 25 degrees Celsius for a period of 8 months with transfer pump agitation in the absence of oxygen and carbon dioxide.

### Example 2

SM and SSFM fermentation of lycopene necessitated the use of the following cultures and reagents: Paracoccus dentrificans inoculant 52.8 ml at 7.385×10⁶ cfu/mL; Streptomyces spp. 28.8 mL at 2.5×10⁶ cfu/mL; vegetable peptone; dextrose; soy extract; agar; and distilled water.

Additionally, the fermentation utilized the following procedures: SSFM agar was prepared by weighing 1.5 g of vegetable peptone, 0.5 g agar and 0.016 g Citro Bio into a 250 mL Erlenmeyer flask and diluting to 100 mL with distilled water. The mixture was boiled for 2 minutes and distributed into 10 separate test tubes, 10 mL/tube, and sterilized with steam for 30 minutes. At the end of sterilization process the tubes were allowed to cool to room temperature prior to inoculating each with 5.2 mL of P. dentrificans 48 hour old culture. The tubes were incubated under 1700 lumens of UV light at 37 degrees Celsius for a period of 6 months. After this period, a “hypersensitized” strain of P. dentrificans was isolated as a dark red colony on a nutrient agar plate. Once isolated this strain was evaluated by HPLC analysis and found to contain a concentration of 7% lycopene, percent per dry weight of the fermentation solids.

Then the organism was streaked, using a whole streak plating technique, onto 100 sterile Petri plates containing 10 mL of the following agar: 10 g/L soy digested soy protein extract, 3.0 g/L dextrose and 20 g/L agar. The plates were inverted and incubated for a period of 48 hours under 1700 lumens of UV Light at 37 degrees Celsius.

At the end of the incubation period the bacterial solids present on the 100 plates was collected in 1 L of sterile peptone water to charge the SM fermentation system. The SM fermentation system was loaded with 10 kg of macro-nutrient spheres having the same formula as those provided for the Petri plates previously described. The SM fermentation was incubated for a period of 48 hours under 1700 lumens of UV light at 37 degrees Celsius. At the end of the incubation period, the system was charged with 1000 mL of Streptomyces spp. before culture and incubation in the dark at 37 degrees Celsius for a period of 8 hours. At the end of the incubation period the system was pressurized with nitrogen gas. This resulted in 1.2 kg of 10% lycopene oil collected. Then 50 g of dispersion oil (having the formula: 99% Tween20, 0.4% vitamin E and 0.6% palmitic acid) was added and the mixture was homogenized at 1300 rpm for a period of 15 minutes.

### Example 3

SM and SSFM fermentation of astaxanthin utilized the following procedures: 100 mL of peptone water was prepared and distributed into 10 test tubes as in Example 1. The tubes were inoculated with 1,000,000 CFU of wild-type Phaffia rhodozyma and incubated at 25 degrees Celsius under 1700 lumen of UV light for a period of 8 months. A bright purple colony was isolated and streaked to 100 Petri dishes, as in Example 1, containing 10 mL of medium (having the formula: 3.0 g/L dextrose, 5.0 g/L yeast extract, 3.0 g/L malt extract and 20 g/L agar). This same formula was used to prepare the macro-nutrient spheres and SM fermentation was conducted as in Example 1. The culture was incubated at 25 degrees Celsius, and the process yielded 1.12 kg of 10% astaxanthin oil.

### Example 4

SM and SSFM fermentation of vitamin K2 MK-7 utilized the following procedures: 100 mL of peptone water was prepared as in Example 1 and distributed into 10 test tubes. The tubes were inoculated with 3,000,000 CFU of wild-type Bacillus subtilis and incubated at 37 degrees Celsius in the dark for a period of 4 months. At the end of the 4-month period the organism was streaked to agar plates (having the formula: 15 g/L soy extract, 10 g/L dextrose and 20 g/L agar). The plates were incubated at 37 degrees Celsius for a period of 48 hours and collected in 1 L of medium which was used to inoculate 151 L of medium containing the above formula minus the agar. This mixture was incubated in the dark at 37 degrees Celsius for a period of 48 hours under gentle agitation (200 rpm), yielding 1.8 kg of 7% vitamin K2 menaguinone 7 powder.

### Example 5

The agar nutrient macro-sphere for a “hypersensitized” strain of Phaffia has the following formula: 3.0 g/L maltose extract, 5.0 g yeast extract, 3.0 g/L dextrose and 20.0 g/L agar. These ingredients are mixed together in an atomization vessel with IL/L distilled water, boiled for 2 minutes under mild agitation and then steam sterilized for a period of 30 minutes. At the end of the sterilization process, the hot solution is rapidly sprayed through an atomization tip 30 into a pool of ionized cold water 32. Upon contacting the surface of the water macro-spheres of agar nutrient are formed 34 (FIG. 2).

1. A method of solid mutagenesis fermentation comprising:
identifying a host source of a lipid-soluble vitamin or nutrient;
producing nutrient macro-spheres;
loading the macro-spheres into a bioreactor;
activating said solid mutagenesis fermentation process within said bioreactor;
separating fermentation solids from the macro-spheres; and
purifying the fermentation solids.

2. The method of claim 1 wherein the host source is botanical.

3. The method of claim 1 wherein the host source is microbial.

4. The method of claim 3 wherein the microbe is selected from the group consisting of Phaffia rhodozyma, Rhodotorula glutinis, Paracoccus, Bacillus subtilis, Fusarium, Trichoderma, Penicillum, Aspergillus, Bacillus licheniformis, Bacillus pumilius, Leuconostoc mesenteroides, Chrysosporium pannorum, Rhizobium and Bradyrhizobium.

5. The method of claim 1 wherein the fermentation solids are selected from the group of consisting of astaxanthin, beta-carotene, lycopene, lutein, coenzyme Q-10, glucosamine, and vitamin K2 MK-7.

6. The method of claim 1 wherein the bioreactor is operated under anaerobic conditions.

7. The method of claim 1 wherein the bioreactor is operated under aerobic conditions.

8. The method of claim 1 wherein the bioreactor is equipped with computerized monitoring devices for the measurement and control of members selected from the group consisting of temperature, pH, carbon dioxide concentration, oxygen concentration, lighting cycles, incubation period, nutrient concentration, nutrient replenishment, and combinations of the same.

9. The method of claim 6 wherein the bioreactor is fully automated.

10. The method of claim 1 wherein the production of nutrient macro-spheres is through sterilization and cold water atomization of an agar nutrient pool.

11. The method of claim 1 further comprising charging the bioreactor with a pool of hypersensitized microbes.

12. The method of claim 1 further comprising altering the genetic code of the host source to redirect cellular activity to the concentration of fermentation solids.

13. The method of claim 11 further comprising stimulating transcription of the host source's genes involved in bioactive expression within the growth phase of the cell cycle.

14. The method of claim 1 wherein the separating of the fermentation solids is caused by purging the system with a gas.

15. The method of claim 1 further comprising undergoing steps of semi-solid fluid mutagenesis.

16. A method of semi-solid fluid mutagenesis fermentation comprising:
identifying a host source of a lipid-soluble vitamin or nutrient;
preparing an agar source;
inoculating the host source in the agar;
vortexing the host source;
incubating the host source;
activating said semi-solid fluid mutagenesis fermentation process within the incubated host source to produce fermentation solids; and
separating fermentation solids.

17. The method of claim 16 wherein the host source is botanical.

18. The method of claim 16 wherein the host source is microbial.

19. The method of claim 18 wherein the microbe is selected from the group consisting of Phaffia rhodozyma, Rhodotorula glutinis, Paracoccus, Bacillus subtilis, Fusarium, Trichoderma, Penicillum, Aspergillus, Bacillus licheniformis, Bacillus pumilius, Leuconostoc mesenteroides, Chrysosporium pannorum, Rhizobium and Bradyrhizobium.

20. The method of claim 16 wherein the fermentation solids are selected from the group consisting of astaxanthin, beta-carotene, lycopene, lutein, coenzyme Q-10, glucosamine and vitamin K2 MK-7.

21. The method of claim 16 further comprising altering the genetic code of the host source to redirect cellular activity to the concentration of fermentation solids.

22. The method of claim 17 further comprising stimulating transcription of the host source's genes involved in bioactive expression within the growth phase of the cell cycle.

23. The method of claim 16 further comprising purifying the fermentation solids.

24. The method of claim 16 further comprising undergoing steps of solid mutagenesis.

25. A method of mutagenesis fermentation to increase fermentation solid yields of a vitamin or nutrient source, comprising:
analyzing a host source of a lipid-soluble vitamin or nutrient for characteristics selected from the group consisting of genetic, biochemical, physical, propagation and combinations of the same;
determining a preferred form of mutagenesis for the host source comprising solid mutagenesis fermentation and/or semi-solid fluid mutagenesis fermentation;
exposing the host source to the fermentation;
generating a hypersensitive strain of the host source; and
producing an increase in fermentation solid yields from the host source.

26. The method of claim 24 further comprising separating the fermentation solid yields.

27. The method of claim 24 further comprising purifying the fermentation solid yields.

28. The method of claim 24 wherein the fermentation solid yields are lipid soluble vitamins or nutrients.

29. The method of 24 wherein generating a hypersensitive strain of the host source further comprises mutating genetic transcriptional control.
